(19) United States
(12) Patent Application Publication
(10) Pub. No.: US 2005/0058731 A1
(43) Pub. Date: Mar. 17, 2005

(54) Antimicrobial composition comprising soy oil, rice bran oil and jojoba oil

(76) Inventor: Daniel J. Beaurline, Dallas, TX (US)

Correspondence Address: SIDLEY AUSTIN BROWN & WOOD LLP, 717 NORTH HARWOOD, SUITE 3400, DALLAS, TX, 75201, US

(21) Appl. No.: 10/497,252

(22) PCT Filed: Nov. 19, 2002
(86) PCT No.: PCT/US02/37161

Related U.S. Application Data
(60) Provisional application No. 60/334,512, filed on Nov. 30, 2001.

(51) Int. Cl. A61K 35/78; C11B 5/00

(52) U.S. Cl. 424/736; 424/750; 424/757; 424/764; 424/745; 424/769; 424/770; 554/2

(57) ABSTRACT

An embodiment of the present invention, including a treatment for cuticles or other skin tissue comprising a mixture of soy oil, rice bran oil, jojoba oil, and an effective amount an antifungal/antimicrobial agent is disclosed. In addition, in a preferred embodiment of the present invention, a treatment for cuticles or other skin tissue comprising a mixture of soy oil, rice bran oil, jojoba oil, avocado oil, primrose oil, juniper oil, grapefruit oil, chamomile oil, lavender oil with an effective amount of an antifungal/antimicrobial agent is described.

### FIELD OF THE INVENTION

The invention relates to a composition that can be applied to human skin and nails, e.g., to the cuticles of fingernails and toenails comprising a broad spectrum antifungal/antimicrobial agent.

### BACKGROUND

Various kinds of compositions for nail application are known, including nail polishes, nail polish removers, nail oil emulsions, cuticle treatments, among others. These compositions range from the purely cosmetic to the primarily medicinal. Oil-containing compositions have been identified as nail and cuticle conditioners or softeners. For example, U.S. Pat. No. 2,765,257 discloses that sulfonated mineral oil acts as an effective cuticle softener, while sulfonated animal and vegetable oils are not effective. Additionally, U.S. Pat. No. 4,286,609 discloses that soaking fingertips in a hot, aqueous emulsion of vegetable and animal oils moisturize, smooth and soften the surface of the nails and the tissue surrounding the nails.

The use of cuticle oil treatments has increased with the growth in popularity of artificial nails and nail sculpturing. The sequential use of oil compositions for nail and cuticle conditioning with the application of artificial nails creates a conducive environment for fungi and bacterial growth around nails because these treatments enhance moisture retention in nails. Consequently, nail sculpturing and applying artificial nails are accompanied by an increase in the incidence of fungal and bacterial infestations and/or infections in nails and surrounding tissue.

U.S. Pat. No. 3,382,151 describes an aqueous-based, formaldehyde-containing composition which can be applied to fingernails to prevent cracking and splitting. The composition of U.S. Pat. No. 3,382,151 possesses aseptic properties, healing inflammation of the nail matrix and killing nail-infested fungi. Treating fungal and bacterial nail infestations and infections with an aqueous-based formaldehyde-containing composition is not completely satisfactory. Repeated use of such compositions, while potentially remedying the fungus infection, oftentimes harms the nails or contiguous area of the skin due to the formaldehyde.

U.S. Pat. RE 36,253 describes a nail and cuticle conditioning composition containing jojoba oil and tolnaftate. The tolnaftate reportedly confers anti-fungal properties to this composition.

Dyer, U.S. Pat. No. 6,022,549, teaches including an antimicrobial agent in a nail care composition. Specifically, Dyer discloses the inclusion of certain benzyl alkonium chlorides and phenols such as resorcinols and cresols in the range of 0.5 percent by weight to about 5 percent by weight.

Sun, et al. in U.S. Pat. No. 6,231,875 disclose a topical composition for treating human nail and skin diseases including fungal infections, bacterial infections, and psoriasis. The composition is comprised of at least one active agent, at least one acidifier, and at least one volatile solvent. One active agent may be triclosan, preferred acidifiers are 10% HCl or 37% HCl, and preferred volatile sovents include ethyl alcohol, isopropyl alcohol, ethyl acetate, and butyl acetate.

However, the use of toxic or harsh chemicals, such as formaldehyde and acids, can be detrimental to the nail and the surrounding tissue. Thus, it is an object of the present invention to provide a nail oil composition which provides a safe and effective remedy for, or prevention against, fungal and bacterial infestation or infection in nails and surrounding tissue.

### SUMMARY OF THE INVENTION

A composition according to the present invention comprises a mixture of soy oil, rice bran oil, jojoba oil, and an effective amount of a broad spectrum antibacterial/antimicrobial agent. The preferable antifungal/antimicrobial agent is 2,4,4′-trichloro-2′-hydroxy-diphenyl-ether, generically referred to as triclosan and sold in the United States under the trade name Irgasan DP-300 by Ciba Specialty Chemicals. A presently preferred composition further includes avocado oil, primrose oil, juniper essential oil, grapefruit essential oil, chamomile oil, and lavender essential oil. This antifungal/antimicrobial composition is preferably used as a cuticle treatment, although it may be applied to other skin tissues as well.

### DETAILED DESCRIPTION OF THE INVENTION

The terms “weight percent” or “percent by weight” herein refer to the percent of a particular component in a composition based upon the total weight of the composition. These terms may be abbreviated as “wt %” or “% wt.” The terms “volume percent” or “percent by volume” herein refer to the percent of a particular component in a composition, based upon the total volume of the composition. These terms may be abbreviated as “v %” or “% v.”

The composition of the invention is nontoxic, comprised only of natural oils and triclosan. The safety of the cuticle oil is important because of the repeated use often necessary with cuticle oils. Additionally, the nontoxicity will be appreciated by consumers concerned about accidental ingestion. Therefore, the composition of the present invention is advantageous over compositions of the prior art that may contain organic solvents or acids. In addition, although the preferred method of using the composition of the present invention is to treat cuticles, the composition can be advantaeously applied to other skin tissues.

Soybean oil is commercially available from Natural Oils International in Arletta, Calif., as “Salad Soybean Oil.” Soybean or soy oil is light, readily absorbed by the skin, and rich in fatty acids and Vitamin E. Any suitable amount of soybean oil is contemplated in this invention. In general, the amount of soybean oil to be incorporated into the composition of the present embodiment of the invention is 25-75% by weight of the total composition. The preferred range of the amount of soybean oil to be incorporated into the composition of the present embodiment of the invention is 40-60% by weight of the total composition.

Rice bran oil (Oryza sativa), which is commercially available from Natural Oils International, is rich in Vitamin E and readily penetrates the skin. Any suitable amount of rice bran oil is contemplated in this invention. In general, the amount of rice bran oil to be incorporated into the composition of the present embodiment of the invention is 25-75% by weight of the total composition. The preferred range of the amount of rice bran oil to be incorporated into the composition of the present embodiment of the invention is 40-60% by weight of the total composition.

Jojoba oil (Simmondsia Chitensis) is commercially available from Purcell Jojoba International as “PNJ Golden Jojoba.” Jojoba oil is a natural cellular renewal ingredient and a moisturizer. Any suitable amount of jojoba oil is contemplated in this invention. In general, the amount of jojoba oil to be incorporated into the composition of the present embodiment of the invention is 0.1-10% by weight of the total composition. The preferred range of the amount of jojoba oil to be incorporated into the composition of the present embodiment of the invention is 1-5% by weight of the total composition.

Avocado oil is expeller pressed, refined, bleached, deodorized and winterized. Such avacodo oil is commercially available from Natural Oil International. Avocado oil readily penetrates the skin, is rich in vitamins and minerals, and conditions and moisturizes the skin. Any suitable amount of avocado oil is contemplated in the present embodiment of this invention. In general, the amount of avocado oil to be incorporated into the composition of the present embodiment of the invention is 0.1-10% by weight of the total composition. The preferred range of the amount of avocado oil to be incorporated into the composition of the present embodiment of the invention is 1-5% by weight of the total composition.

Primrose oil is cold pressed. Such primrose oil is commercially available from Natural Oils International as “Evening Primrose Oil” (Enothera Biennis). Any suitable amount of primrose oil is contemplated in the present embodiment of the invention. In general, the amount of primrose oil to be incorporated into the composition of the present embodiment of the invention is 0.1-10% by weight of the total composition. The preferred range of the amount of primrose oil to be incorporated into the composition of the present embodiment of the invention is 1-5% by weight of the total composition.

Juniper oil used in the present embodiment of the invention is commercially available from Carnazzi Essential Oil Therapy in Santa Ynez, Calif., and is sold as “Juniper Berry Wild Himalayan Oil.” Any suitable amount of juniper oil is contemplated in the present embodiment of the invention. In general, the amount of juniper oil to be incorporated into the composition of the present embodiment of the invention is 0.01-0.5% by weight of the total composition. The preferred range of the amount of juniper oil to be incorporated into the composition of the present embodiment of the invention is 0.1-0.3% by weight of the total composition.

Lavender oil (Lavendila Officialis) used in the present embodiment of the invention is commercially available from Carnazzi Essential Oil Therapy under the name “Lavender 40/42.” Lavender Oil promotes healing acting as an antiseptic, analgesic, antibiotic, and bactericide. Lavender oil also reduces scarring and stimulates growth of new cells. Any suitable amount of lavender oil is contemplated in the present embodiment of the invention. In general, the amount of lavender oil to be incorporated into the composition of the present embodiment of the invention is 0.001-0.1% by weight of the total composition. The preferred range of the amount of lavender oil to be incorporated into the composition of the present embodiment of the invention is 0.01-0.5% by weight of the total composition.

Chamomile oil (Oremensis Multicazilis) is commercially available from Carnazzi Essential Oil Therapy as “Chamomile Maroc.” Any suitable amount of chamomile oil is contemplated in the present embodiment of the invention. In general, the amount of chamomile oil to be incorporated into the composition of the present embodiment of the invention is 0.001-0.1% by weight of the total composition. The preferred range of the amount of chamomile oil to be incorporated into the composition of the present embodiment of the invention is 0.01-0.5% by weight of the total composition.

Grapefruit oil used in the present invention is commercially available from Carnazzi Essential Oil Therapy as “Grapefruit White.” Any suitable amount of grapefruit oil is contemplated in the present embodiment of the invention. In general, the amount of grapefruit oil to be incorporated into the composition of the present embodiment of the invention is 0.01-0.5% by weight of the total composition. The preferred range of the amount of grapefruit oil to be incorporated into the composition of the present embodiment of the invention is 0.1-0.3% by weight of the total composition.

The triclosan (2,4,4′-trichloro-2′-hydroxy-diphenyl-ether) used in the present embodiment of the invention is commercially available from Ciba Specialty Chemicals under the trade name Irgasan DP-300. A sufficient amount of triclosan is used so as to be an effective antifungal/antimicrobial agent. In general, the amount of triclosan employed is in the range of 0.5-5% by weight of the total composition. The preferred range of the amount of triclosan to be incorporated into the composition of the present embodiment of the invention is 1-3% by weight of the total composition.

### EXAMPLE I

In one embodiment of the present invention, the oils and antifungal/antimicrobial agent are mixed together in the quantities listed below.

| | Quantity in | Percent | Quantity in | Percent |
|---|---|---|---|---|
| | Ounces per | By | Pounds per | by |
| | One Gallon | Volume | One Gallon | Weight |
| Soybean Oil | 56.32 | 43.49 | 3.37 | 43.21 |
| Rice Bran Oil | 56.32 | 43.49 | 3.37 | 43.21 |
| Jojoba Oil | 5.12 | 4.0 | 0.29 | 3.69 |
| Avocado Oil | 5.12 | 4.0 | 0.29 | 3.69 |
| Primrose Oil | 5.12 | 4.0 | 0.29 | 3.69 |
| Juniper Essential Oil | 0.25 | 0.195 | 0.014 | 0.181 |
| Grapefruit Essential Oil | 0.165 | 0.129 | 0.0091 | 0.117 |
| Chamomile Oil | 0.022 | 0.018 | 0.0031 | 0.017 |
| Lavender Essential Oil | 0.011 | 0.009 | 0.00062 | 0.008 |
| Triclosan | 1.52 | 1.186 | 0.156 | 2.00 |

The composition is prepared by charging first the soybean oil in a mixing vessel under low agitation. Then, the remaining above listed oils are added to the mixing vessel under low agitatation. The triclosan, in solid form, is then added to the agitated oil solution and the mixture is further agitated until the solid has completely dissolved. The resulting composition can then be topically applied to the finger and toe nails of a user. In addition to the preferred method of using the composition of the present invention, one skilled in the relevant art would also recognize that the composition can also be applied to other skin tissues.

Although specific embodiments of the invention are described herein, they are not to be interpreted as limiting the scope of the present invention. Many other embodiments will become apparent to those skilled in the art in light of the teachings of this written description. The scope of the invention is only limited by the claims appended hereto.

1. A composition comprising a mixture of soy oil, rice bran oil, jojoba oil, and an effective amount of an antifungal/antimicrobial agent.

2. A composition of claim 1, further comprising avocado oil.

3. A composition of claim 1, further comprising primrose oil.

4. A composition of claim 1, further comprising juniper oil.

5. A composition of claim 1, further comprising grapefruit oil.

6. A composition of claim 1, further comprising chamomile oil.

7. A composition of claim 1, further comprising lavender oil.

8. A composition of claim 1, wherein said antifungal/antimicrobial agent is triclosan.

9. A composition of claim 1, wherein said antifungal/antimicrobial agent is present in the range of 0.5 to 5 percent by weight of the mixture.

10. A composition of claim 1, wherein said antifungal/antimicrobial agent is present in the range of 1 to 3 percent by weight of the mixture.

11. A composition of claim 1, wherein said antifungal/antimicrobial agent is 2.0 percent by weight of the mixture.

12. A composition of claim 1, wherein said soy oil is present in a range of 25-75% by weight of the mixture.

13. A composition of claim 1, wherein said soy oil is present in a range of 40-60% by weight of the mixture.

14. A composition of claim 1, wherein said rice bran oil is present in a range of 25-75% by weight of the mixture.

15. A composition of claim 1, wherein said rice bran oil is present in a range of 40-60% by weight of the mixture.

16. A composition of claim 1, wherein said jojoba oil is present in a range of 0.1-10% by weight of the mixture.

17. A composition of claim 1, wherein said jojoba oil is present in a range of 1-5% by weight of the mixture.

18. A composition of claim 2, wherein said avocado oil is present in a range of 0.1-10% by weight of the mixture.

19. A composition of claim 2, wherein said avocado oil is present in a range of 1-5% by weight of the mixture.

20. A composition of claim 3, wherein said primrose oil is present in a range of 0.1-10% by weight of the mixture.

21. A composition of claim 3, wherein said primrose oil is present in a range of 1-5% by weight of the mixture.

22. A composition of claim 4, wherein said juniper oil is present in a range of 0.01-0.5% by weight of the mixture.

23. A composition of claim 4, wherein said juniper oil is present in a range of 0.1-0.3% by weight of the mixture.

24. A composition of claim 5, wherein said grapefruit oil is present in a range of 0.01-0.5% by weight of the mixture.

25. A composition of claim 5, wherein said grapefruit oil is present in a range of 0.1-0.3% by weight of the mixture.

26. A composition of claim 6, wherein said chamomile oil is present in a range of 0.001-0.1% by weight of the mixture.

27. A composition of claim 6, wherein said chamomile oil is present in a range of 0.01-0.5% by weight of the mixture.

28. A composition of claim 7, wherein said lavender oil is present in a range of 0.001-0.1% by weight of the mixture.

29. A composition of claim 7, wherein said lavender oil is present in a range of 0.01-0.5% by weight of the mixture.

30. A method to treat a nail cuticle comprising applying to said cuticle an oil composition comprising triclosan.

31. A method of claim 30, wherein said oil composition comprises a mixture of soy oil, rice bran oil, and jojoba oil.

32. A method of claim 31, wherein said oil composition further comprises avocado oil.

33. A method of claim 31, wherein said oil composition further comprises juniper oil.

34. A method of claim 31, wherein said oil composition further comprises grapefruit oil.

35. A method of claim 31, wherein said oil composition further comprises chamomile oil.

36. A method of claim 31, wherein said oil composition further comprises lavender oil.
